(19) United States
(12) Patent Application Publication
(10) Pub. No.: US 2013/0123657 A1
(43) Pub. Date: May 16, 2013

(54) THERMOCHROMATIC EARPLUG AND METHOD FOR MANUFACTURING THE SAME

(71) Applicant: Koo Ja Heon, Goyang-si (KR)

(72) Inventor: Koo Ja Heon, Goyang-si (KR)

(21) Appl. No.: 13/335,351

(22) Filed: Dec. 22, 2011

(30) Foreign Application Priority Data
Nov. 15, 2011 (KR) 10-2011-0118721

(51) Int. Cl. A61B 5/01; B29C 39/36; B29C 39/42

(52) U.S. Cl. 600/549; 264/102; 264/334

(57) ABSTRACT

Disclosed are a thermochromatic earplug and a method for manufacturing the same, in which the color of the thermochromatic earplug is changeable according to the body temperature of a wearer, so that the health state of the wearer is able to be determined through the color of the thermochromatic earplug. The method for manufacturing the thermochromatic earplug includes (a) mixing an earplug raw material including urethane resin with a thermochromatic material, (b) molding and curing a mixture, which is obtained by mixing the earplug raw material with the thermochromatic material, into an earplug shape by pouring the mixture into a mold having a cavity with a shape corresponding to the earplug shape, and (c) demolding a resultant structure, which is obtained by molding and curing the mixture into the earplug shape, from the mold.

### CROSS-REFERENCE TO RELATED APPLICATION

This application claims the benefit under 35 U.S.A. §119 of Korean Patent Application No. 10-2011-0118721, filed on Nov. 15, 2011 in the Korean Intellectual Property Office, the contents of which is incorporated herein by reference in its entirety.

### BACKGROUND OF THE INVENTION

1. Field of the Invention

The present invention relates to an earplug. In more particular, the present invention relates to a thermochromatic earplug and a method for manufacturing the same, in which the color of the thermochromatic earplug is changeable according to the body temperature of a wearer, so that the health state of the wearer can be determined through the color of the thermochromatic earplug.

2. Description of the Related Art

An earplug is a tool inserted into an ear of a wearer to keep out external sound. In general, the earplug has been utilized for the purpose of preventing noise.

Recently, many researches and studies on the earplug have been performed.

Korean Registered Utility Mode Publication No. 20-0276640 (published on May 25, 2002) discloses an earplug for preventing noise. In the earplug for preventing noise disclosed in the cited reference, a wearer can feel stability and the earplug does not get out of the ear of the wearer when the wearer wears the earplug, by improving the shape of the earplug.

In addition, Korean Unexamined Patent Publication No. 10-2011-0101963 (published on Sep. 16, 2011) discloses an earplug representing a high soundproof property. The earplug representing the high soundproof property disclosed in the cited reference has a size greater than a size of an ear hole of a wearer, and air is injected into the earplug. Accordingly, when the earplug is inserted into to the ear of the wearer, the earplug can be compressed, thereby improving soundproof and waterproof effects.

As shown in the cited references, researches and studies on the earplug have been performed while focusing on the improvement of the wearability and the ability of preventing noise of the earplug by improving the shape or the structure of the earplug.

### SUMMARY OF THE INVENTION

An object of the present invention is to provide a thermochromatic earplug and a method for manufacturing the same, in which the color of the earplug is changeable according to the body temperature of a wearer, so that the health state of the wearer can be determined through the color of the earplug.

In order to accomplish the object of the present invention, according to an aspect of the present invention, there is provided a method for manufacturing a thermochromatic earplug, which includes (a) mixing an earplug raw material including urethane resin with a thermochromatic material, (b) molding and curing a mixture, which is obtained by mixing the earplug raw material with the thermochromatic material, into an earplug shape by pouring the mixture into a mold having a cavity with a shape corresponding to the earplug shape, and (c) demolding a resultant structure, which is obtained by molding and curing the mixture into the earplug shape, from the mold.

The thermochromatic material includes reversible thermochromatic pigments.

In step (a), the mixture, which is obtained by mixing the earplug raw material with the thermochromatic material, is vaccum-defoamed in a vacuum defoamer. In addition, in step (a), 5 to 35 parts by weight of the thermochromatic material are mixed with the earplug raw material based on 100 parts by weight of the urethane resin.

In addition, the earplug raw material preferably includes 100 parts by weight of isocyanate solution representing viscosity of 150 cps to 450 cps at a temperature of 40° C., and 190 to 210 parts by weight of polyol solution representing viscosity of 100 cps to 350 cps at the temperature of 40° C.

In addition, step (b) is performed at a temperature of about 60° C. to about 80° C.

In order to accomplish the above object of the present invention, according to another aspect of the present invention, there is provided a thermochromatic earplug including urethane resin and a thermochromatic material. The thermochromatic earplug represents a first color at less than a preset temperature, and represents a second color different from the first color at the preset temperature or more.

In addition, the urethane resin has free rise density of 0.13 g/cc to 0.14 g/cc.

According to the thermochromatic earplug of the present invention, when the body temperature of a wearer is increased, the color of the thermochromatic earplug can be changed. Accordingly, the health state of the wearer of the thermochromatic earplug can be determined through the color of the thermochromatic earplug.

### BRIEF DESCRIPTION OF THE DRAWINGS

FIG. 1 is a view showing an example of the shape of a thermochromatic earplug according to the present invention; and

FIG. 2 is a flowchart schematically showing a method for manufacturing a thermochromatic earplug according to the present invention.

### DETAILED DESCRIPTION OF THE INVENTION

The advantages, the features, and schemes of achieving the advantages and features will be apparently comprehended by those skilled in the art based on the embodiments, which are detailed later in detail, together with accompanying drawings. The present invention is not limited to the following embodiments but includes various applications and modifications. The embodiments will make the disclosure of the present invention complete, and allow those skilled in the art to completely comprehend the scope of the present invention.

Hereinafter, a thermochromatic earplug 150 and a method for manufacturing the same according to the present invention will be described in detail with reference to accompanying drawings.

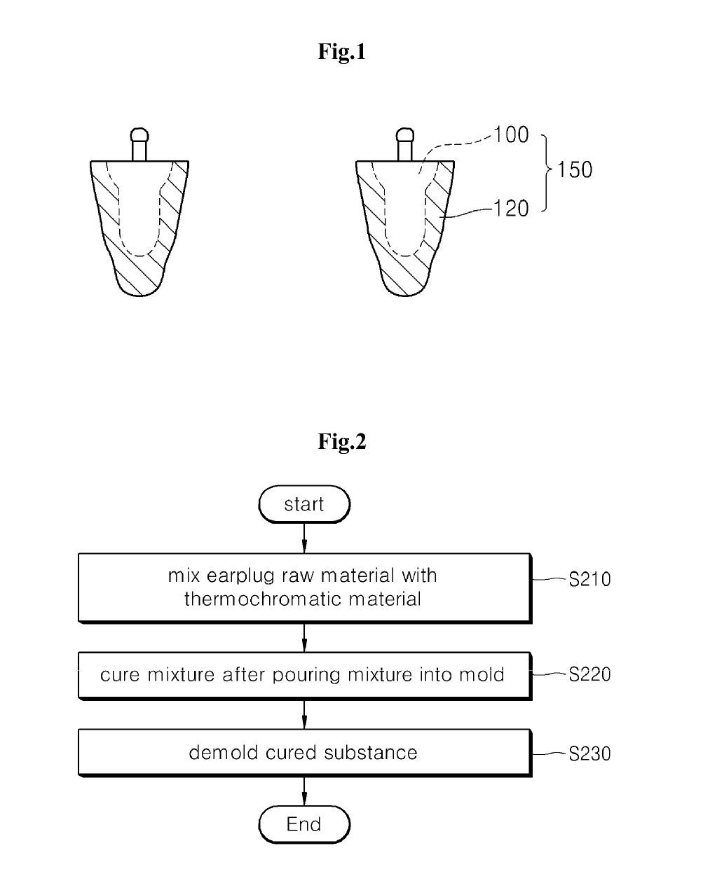

FIG. 1 is a view showing an example of the shape of the thermochromatic earplug 150 according to the present invention.

The thermochromatic earplug 150 shown in FIG. 1 includes an inner tip 100 and an outer tip 120 according to an embodiment of the present invention.

The inner tip 100 serves as a core, and forms the framework of the thermochromatic earplug 150. The inner tip 100 may include a material selected from the group consisting of polyethylene, acrylonitrile butadiene styrene copolymer, polypropylene, polystyrene, urethane rubber, and rigid polyurethane.

If necessary, the inner tip 100 may not be used. However, the inner tip 100 is preferably used in order to maintain the shape of the thermochromatic earplug 150.

The outer tip 120 serves as a body of the thermochromatic earplug 150. The outer tip 120 coupled with the inner tip 100 may be inserted into the ear of a wearer, or the outer tip 120 itself may be inserted into the ear of the wearer. The outer tip 120 may include flexible polyurethane. The inner tip 100 may be coupled with the outer tip 120 by using an adhesive agent. In addition, the inner tip 100 may be coupled with the outer tip 120 without the adhesive agent by molding the outer tip 120 in the state that the inner tip 100 is previously fixed into a mod for the outer tip 120.

FIG. 2 is a flowchart schematically showing a method for manufacturing the thermochromatic earplug 150 according to the present invention.

Referring to FIG. 2, the method for manufacturing the thermochromatic earplug 150 includes a raw material mixing step (step S210), a molding and curing step (S220), and a demolding step (step S230).

In the raw material mixing step (step S210), an earplug raw material including urethane resin is mixed with a thermochromatic material to form a mixture.

The thermochromatic material may include reversible thermochromatic pigments.

In this case, after the earplug raw material has been mixed with the thermochromatic material, the mixture is vaccum-defoamed in a vacuum defoamer so that the soundproof characteristic of the manufactured thermochromatic earplug 150 can be improved.

In this case, preferably, 5 to 35 parts by weight of the thermochromatic material are mixed with the earplug raw material based on 100 parts by weight of the urethane resin. If less than 5 parts by weight of the thermochromatic material is used based on 100 parts by weight of the urethane resin, the thermochromatic effect of an earplug to be manufactured may be insufficiently represented. In contrast, if the usual dose of the thermochromatic material exceeds 35 parts by weight based on 100 parts by weight of the urethane resin, the hardness of the earplug to be manufactured is increased, so that the wearability of the earplug may be degraded. In addition, after a wearer wears the earplug, the earplug is not fully filled in the ear hole of the wearer, so that the effect of preventing noise may be degraded.

Preferably, the earplug raw material preferably contains 100 parts by weight of isocyanate solution representing the viscosity of 150 cps to 450 cps at the temperature of 40, and 190 to 210 parts by weight of polyol solution representing the viscosity of 100 cps to 350 cps at the temperature of 40. If the usual dose of the polyol solution is less than 190 parts by weight based on 100 parts by weight of the isocyanate solution, the characteristic of preventing noise of the earplug to be manufactured may be degraded. In contrast, if the usual dose of the polyol solution exceeds 200 parts by weight based on 100 parts by weight of the isocyanate solution, the earplug to be manufactured is cured, so that the wearability of the earplug may be degraded.

In the molding and the curing step (step S220), the mixture, which is obtained by mixing the earplug raw material with the thermochromatic material, is put into the mold having cavity corresponding to the earplug to be manufactured, so that the mixture is molded and cured in the form of the earplug. In the present step S220, the molding and curing work is preferably performed for about 5 minutes at the temperature of about 60° C. to about 80° C. When the molding and curing work is performed at the temperature of less than 60° C., the shape of the earplug may not be desirably molded. In contrast, when the molding and curing work is performed at the temperature of 80° C. or more, the earplug may not represent normal performance.

If the composition of the earplug raw material and the conditions of the molding/curing work are satisfied, the free rise density of urethane becomes 0.13 g/cc to 0.14 g/cc, so that the wearability of the earplug and the characteristic of preventing noise can be suitably obtained.

In the demolding step (step S230), a resultant structure molded and cured in the shape of the earplug is demolded from the mold.

The thermochromatic earplug 150 manufactured through the steps S210 to S230 contains urethane resin and thermochromatic material. Accordingly, the thermochromatic earplug 150 represents a first color at less than a preset temperature, and represents a second color different from the first color at the preset temperature or more.

The thermochromatic characteristics can be represented by containing the thermochromatic material together with the earplug raw material when the thermochromatic earplug 15 is manufactured.

The thermochromatic earplug 15 has advantages in the following cases.

For example, if the color of the thermochromatic earplug 15 according to the present invention is set to be changed from yellow to red at the temperature of about 37, and if a wearer of the thermochromatic earplug 15 has a normal body temperature of about 36.5, the color of the thermochromatic earplug 15 is maintained as yellow. However, if the wearer is in a bad health state, so that the body temperature of the wearer is 37 or more, the color of the thermochromatic earplug 15 is changed to red.

In addition, when an infant having high fever falls asleep in the state that the infant wears the thermochromatic earplug 15 according to the present invention, if the color of the thermochromatic earplug 15 is changed from red to yellow, it can be determined that the fever of the infant goes down.

Although a preferred embodiment of the present invention has been described for illustrative purposes, those skilled in the art will appreciate that various modifications, additions and substitutions are possible, without departing from the scope and spirit of the invention as disclosed in the accompanying claims.

What is claimed is:

1. A method for manufacturing a thermochromatic earplug, the method comprising:
(a) mixing an earplug raw material including urethane resin with a thermochromatic material;
(b) molding and curing a mixture, which is obtained by mixing the earplug raw material with the thermochromatic material, into an earplug shape by pouring the mixture into a mold having a cavity with a shape corresponding to the earplug shape; and
(c) demolding a resultant structure, which is obtained by molding and curing the mixture into the earplug shape, from the mold.

2. The method of claim 1, wherein, in step (a), the mixture, which is obtained by mixing the earplug raw material with the thermochromatic material, is vaccum-defoamed in a vacuum defoamer.

3. The method of claim 1, wherein the thermochromatic material includes reversible thermochromatic pigments.

4. The method of claim 1, wherein, in step (a), 5 to 35 parts by weight of the thermochromatic material are mixed with the earplug raw material based on 100 parts by weight of the urethane resin.

5. The method of claim 1, wherein the earplug raw material includes 100 parts by weight of isocyanate solution representing viscosity of 150 cps to 450 cps at a temperature of 40° C., and 190 to 210 parts by weight of polyol solution representing viscosity of 100 cps to 350 cps at the temperature of 40° C.

6. The method of claim 1, wherein step (b) is performed at a temperature of about 60° C. to about 80° C.

7. A thermochromatic earplug including urethane resin and a thermochromatic material, wherein the thermochromatic earplug represents a first color at less than a preset temperature, and represents a second color different from the first color at the preset temperature or more.

8. The thermochromatic earplug of claim 7, wherein the thermochromatic material includes reversible thermochromatic pigments.

9. The thermochromatic earplug of claim 7, wherein 5 to 35 parts by weight of the thermochromatic material are included based on 100 parts by weight of the urethane resin.

10. The thermochromatic earplug of claim 7, wherein the urethane resin has free rise density of 0.13 g/cc to 0.14 g/cc.
